(19) United States
(12) Patent Application Publication
(10) Pub. No.: US 2023/0175934 A1
(43) Pub. Date: Jun. 8, 2023

(54) SAMPLING HOUSING AND MODULAR CONTROLLER COMPRISING THIS SAMPLING HOUSING

(71) Applicant: L'ETAT FRANÇAIS REPRÉSENTÉ PAR LE MINISTÉRE DE L'INTÉRIEUR, Issy-Les-Moulineaux (FR)

(72) Inventors: Amaury PUSSIAU, Cergy Pontoise (FR); Sylvain HUBAC, Cergy Pontoise (FR)

(21) Appl. No.: 17/999,095

(22) PCT Filed: May 20, 2021
(86) PCT No.: PCT/FR2021/050910

§ 371 Date: Nov. 17, 2022

(30) Foreign Application Priority Data
May 20, 2020 (FR) 2005169

(51) Int. Cl. G01N 1/30; G01N 33/483; G01N 33/487; G01N 35/04; B01L 1/00

(52) U.S. Cl. CPC G01N 1/30; G01N 33/4833; G01N 33/487; G01N 35/04; B01L 1/50; B01L 1/52; G01N 2035/0498; G01N 2035/0405; G01N 2035/0458; G01N 2035/00306

(57) ABSTRACT

The present invention relates to a sampling housing and to a modular controller formed by this sampling housing when it is connected to a control station. This housing comprises a device supporting an assembly (3) consisting of a well plate (4), of a cap adapter (5) comprising as many orifices as there are wells and arranged on said well plate (4), and of caps (6), each cap (6) cooperating with a single well of the plate (4). The invention is applicable in the field of analyzing biological samples.

The present invention relates to a sampling housing and to a modular controller formed by this sampling housing when it is connected to a control station.

The technical processes for DNA or RNA type nucleic acid analyses must meet the requirements of the ISO/IEC 15189 standard concerning medical biology laboratories or the ISO/IEC 17025 standard concerning calibration and testing laboratories. The requirements of these standards focus in particular on the fight against operator and sample contamination as well as on sample traceability, from receipt in the laboratory to reporting of the results.

This notion of traceability is all the more critical when laboratories have to analyze several hundreds or thousands of samples per month. To this end, many technical and instrumental solutions have been developed to ensure the quality of the results from the cell lysis step to the analysis results. However, the preliminary stage, called sampling, which consists in positioning a given sample (or a fraction of a sample) in its location for analysis, remains a manual stage that does not present all the necessary guarantees to avoid any risk of sample inversion or contamination. The consequences can be critical in an analysis.

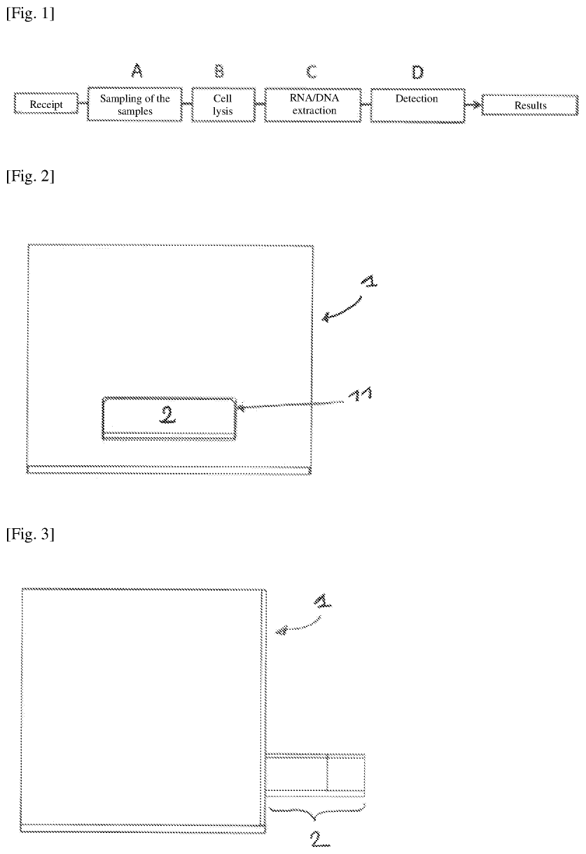

In FIG. 1, the different stages of the conventional process for detecting nucleic acids by molecular biology, for example of the coronavirus SARS-CoV-2 (COVID-19), are shown. The sampling step (A) is manual while all the other steps of the analysis process, namely cell lysis (B), nucleic acid extraction (C) and detection by real-time polymerase chain reaction (RT-PCR) (D), are automated.

The sampling step is all the more critical as it is the entry point for any sample in the analysis chain. Thus, today there is a real need for an integrated device making it possible to ensure the traceability and sampling of biological samples without risk of contamination or risk of inversion.

The sampling of biological samples consists in inserting all or part of a biological sample, solid or liquid, in a location provided for its analysis, for example in an individual tube or in a well of a plate with 96 positions. This sample can be the head of a swab containing the biological element to be analyzed, a solid element such as a fragment of tissue containing genetic material, for example, or a biological fluid directly. This step is followed by cell lysis, which consists in adding reagents that will break the cell membranes to release the nucleic acids (DNA and RNA).

With the appearance of the COVID-19 health crisis and the need to carry out massive screening of the population, medical biology laboratories have massively equipped themselves with “open” nucleic acid extraction systems (e.g.: KingFisher™ Flex system from the company ThermoFisher) that allow them to directly process up to 96 samples in a deep-well plate. The disadvantage is that the sampling must be carried out in an “open” plate instead of individual “closed” tubes.

The use of a 96-well plate therefore does not make it possible to close each location, unlike a tube, which increases the risk of positioning error and inter-well contamination.

This sampling is carried out manually within the plate, and it is not always easy to insert the right sample in the right location and without contamination of the adjacent wells. This step is repetitive when hundreds of samples are analyzed per day and therefore requires the technician to be very attentive to avoid any errors.

Since sampling is the most critical step of the entire analysis process, it must therefore be optimized in order to avoid any risk of sample or operator contamination or of positioning error so as to ensure the quality of the test results. In addition, under certain analysis conditions, it was noted that the sampling of biological samples had to be carried out by regulation under a type 2 biosafety cabinet (BSC). This environment, constrained in terms of space, coupled with a high volume of samples to be analyzed is an obvious source of contamination and traceability errors. To date, there is no sampling device that can be integrated into BSC2 capable of processing a large number of samples in compliance with the requirement of the ISO/IEC 15189 standard.

Document US2018/071731 describes a device in which the wells of a 96-well microplate can be opened exclusively manually in rows of 8 wells. Such a mechanism thus does not make it possible to effectively limit cross-contamination (contamination of adjacent wells) and sample positioning errors during the sampling step. In addition, the opening of the wells is necessarily manual and therefore requires manipulation by the user.

Thus, one of the aims of the present invention is to provide a sampling housing that makes it possible to counter the various drawbacks cited above.

Another object of the invention is to connect this sampling housing to a control station composed of a computer part and an electronic part to form a modular controller.

These aims, as well as others that will appear subsequently, are achieved by a sampling housing for biological samples, solid or liquid, which is, according to the present invention, characterized in that it comprises a device supporting an assembly consisting of a 96-well plate (also called a deep-well plate), a cap adapter comprising as many orifices as there are wells and arranged on this deep-well plate, and caps, each cap cooperating with a single well.

“Sampling housing” refers to a device allowing step A of the conventional process to be carried out for detecting nucleic acids by molecular biology as summarized in FIG. 1, that is to say, a device allowing the efficient sampling of biological samples in different wells in order to be able to proceed with the following steps (B, C, D and results in FIG. 1).

“Operator” refers to a human being carrying out the sampling, for example a laboratory technician.

“Sampling” refers to the action performed by an operator of inserting a biological sample or a sample into one of the wells of the 96-well plate.

“Biological sample” or “sample” refers to a solid or liquid element containing biological material. These biological samples can come from any type of environment, for example from a crime scene, or from a sample taken from a patient as part of COVID-19 screening.

“96-well plate” refers to the plates, also called deep-well plates, comprising wells aligned in 8 rows×12 columns. The device according to the invention can be used with any 96-well plate in the format of the ANSI/SLAS (American National Standards Institute—Society for Laboratory Automation and Screening).

“Device supporting an assembly” means that said device makes it possible to encompass the assembly, making it possible to lock it and hold it during use.

“Cap adapter” refers to a plate making it possible to cover all the wells of a 96-well plate in order to avoid any inter-well contamination, during the insertion into each of these wells of the samples to be analyzed, whether the latter are solid or liquid. The cap adapter according to the invention comprises 96 holes and is designed to be placed above a 96-well plate by being superimposed on the 96 orifices of the well plate receiving the samples. This cap adapter is superimposed on said 96-well plate, and receives the caps on its upper part, allowing the orifices of said plate to be closed, in order to isolate each well of the 96-well plate from the external environment. This avoids any outside contamination. Such an adapter thus allows the use of 96-well plates having wells of different shapes (square, rectangular, circular).

“Cap” refers to an element making it possible to close each well independently. The assembly according to the invention comprises as many caps as wells.

According to a preferred embodiment of the present invention, the sampling housing comprises a drawer supporting the well plate and the cap adapter.

“Drawer” refers to a sliding element in the sampling housing between a position internal to said sampling housing and a position external to said sampling housing.

Advantageously, the sampling housing comprises means for sliding the drawer in said sampling housing that are coordinated with means for removing and replacing one cap of a well at a time.

“Means allowing the drawer to slide” refers to means allowing said drawer to move between its two positions, internal and external to said sampling housing. Preferably, said means is an electric actuator.

According to the invention, “electric actuator” refers to a device making it possible to convert the rotary movement of a motor into linear movement in order to move and position a load, namely the drawer in the case of the invention. Such electric actuators can for example be a guided electric actuator with ball screw drive, for example of the SMC brand of the LEFS series.

According to the invention, “coordinated with means for removing and replacing one cap of a well at a time” means that when the device supporting the assembly, preferably the drawer, slides toward the operator, a single well has been opened by the sampling housing.

Preferably, the drawer slides between a position internal to said sampling housing in which a cap is removed or replaced, and a second position external to said sampling housing in which a sample to be analyzed is introduced into the well whose cap has been removed. Providing a single open well allows the operator to avoid any positioning error when inserting the sample into the plate.

Advantageously, the sampling housing comprises a harpoon for removing and replacing each cap.

“Harpoon” refers to a means of removing a single cap from the assembly.

Preferably, the drawer is composed of two parts: a base to receive the well plate and a removable element making it possible to hold this plate on this base during sampling. The drawer is designed to encompass and isolate the well plate to prevent outside contamination. The design of the drawer, composed of a base and the removable element, also makes it easier to clean and decontaminate the assembly.

According to another aspect, the invention relates to a method for sampling biological samples, solid or liquid, using a modular controller according to the invention comprising the steps of:
a. Scanning a solid or liquid biological sample;
b. Moving means for removing and replacing a cap of a well above a well of the plate;
c. Removing the cap from said well of step b. via said means for removing and replacing a cap of a well;
d. Moving, via the sampling housing, the device (2) supporting the assembly (3) in front of an operator;
e. Depositing, by an operator, of a biological sample in said open well;
f. Moving, via said sampling housing, the device (2) supporting the assembly (3) toward the inside of said sampling housing;
g. Closing, via said means for removing and replacing a cap, the open well comprising a sample by means of its cap.

“Scan of a sample” means the reading by the computer means of said control station of the identifier of a biological sample that was assigned to it when it was taken from a crime scene, or a sample taken from a patient as part of COVID-19 screening,

Preferably, when several biological samples must be sampled, steps a. to g. are repeated in order to successively sample biological samples in different wells, step c. removing a different cap each time.

Before this method, the 96-well plate was arranged in the sampling device according to the invention.

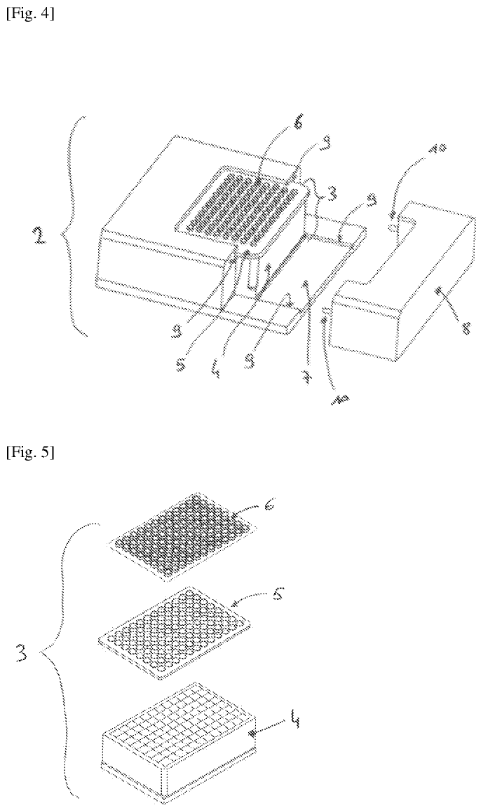

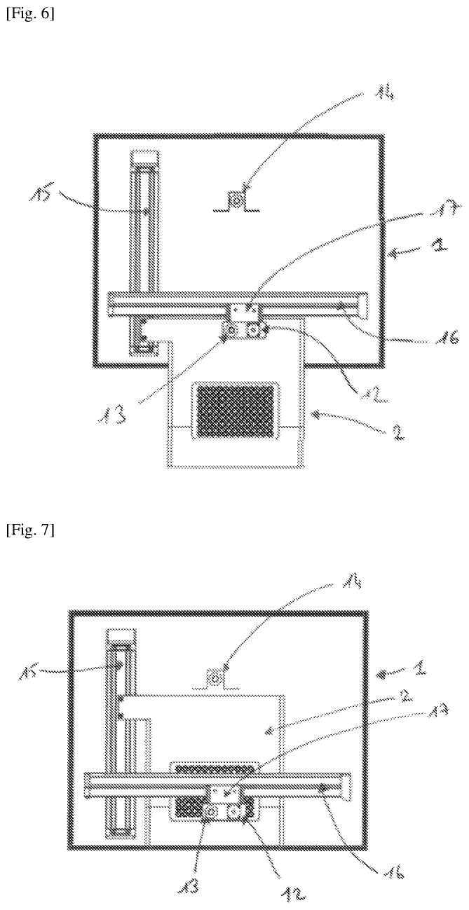

The following description, which is in no way limiting, should be read in conjunction with the appended figures, in which:

[FIG. 1] shows the different steps of the conventional detection process by molecular biology;

[FIG. 2] is a front view of a sampling housing according to the present invention;

[FIG. 3] is a side view of the sampling housing according to FIG. 2 with the drawer in the external position;

[FIG. 4] is a schematic perspective view of the sampling drawer according to one embodiment of the invention;

[FIG. 5] is a perspective view of the assembly of a deep-well plate with cap adapter and caps;

[FIG. 6] is a schematic internal view, seen from above, of the sampling housing with the drawer pulled out;

[FIG. 7] is a schematic internal view, top view, of the sampling housing with the drawer in the closed position.

As can be seen in the figures, according to the present invention a sampling housing 1 comprises a device 2 supporting an assembly 3 consisting of a well plate 4, a cap adapter 5 and caps 6, each cap cooperating with a single orifice of the adapter 5 placed on the well plate 4.

The cap adapter 5 makes it possible to cover all the wells of a 96-well plate 4 in order to avoid any inter-well contamination, during the insertion into each of these wells of the samples to be analyzed, whether the latter are solid or liquid.

The cap adapter 5 has 96 holes and is designed to be placed above a well plate 4 regardless of the manufacturer: the 96 holes of the latter are perfectly superimposed with the 96 orifices of the well plate 4 receiving the samples. The addition of caps 6 then makes it possible to isolate each well of the well plate 4.

With such a device 2, it is possible to perform manual sampling of the well plate 4. It is then necessary to remove a cap 6 using sterile forceps in order to be able to insert the element to be analyzed into the well thus opened. The cap 6 is then replaced in its original location manually using forceps. The risk of contamination is controlled by opening only one well at a time owing to the presence of independent caps 6.

According to an automatable embodiment of the present invention, the device 2 is a drawer that can slide inside and outside the sampling housing 1.

The drawer 2 is designed to accommodate a 96-well plate to the ANSI/SLAS standard, such as a deep-well or PCR plate, for example.

The drawer 2 as well as the cap adapter 5 are made of a material that can be easily decontaminated, such as aluminum or stainless steel.

The sampling housing 1 is composed of the following elements:

a sliding drawer 2 composed of a base 7 allowing reception of any type of 96-well plate 4 (deep-well or PCR plate, for example) and a removable element 8 allowing it to be held on the base 7. The assembly 3 is deposited in this drawer 2 when the element 8 is removed. The assembly 3 is inserted into the drawer 2 using guide rails 9 specific to the cap adapter 5 and the 96-well plate 4 to ensure correct positioning of the assembly 3. The removable element 8 is repositioned on the drawer 2 after insertion of the assembly 3 in order to block the assembly 3 in the drawer 2. The removable element 8 comprises guide rods 10 to ensure its correct positioning on the drawer 2. The use of guide rails 9 makes it possible to slide the assembly 3 in the drawer without exerting any particular pressure.

an orifice 11 on its front facade (i.e. the one facing the operator) that is adjusted to the shape of the drawer 2, preventing the operator from any risk of injury when closing the drawer 2.

a means for removing and replacing each cap 6, such as a harpoon 12, in order to then allow the deposit of a sample in the well in question. The removed cap remains hooked to the harpoon during the phases of opening the drawer, sampling a sample, and closing the drawer.

It can also comprise:

a vertical camera 13 making it possible to take a photograph of each well of the plate 4. This functionality makes it possible to ensure that a cap 6 is indeed present before opening, that the well of interest is indeed empty before sampling, then filled with a biological sample, and that the cap 6 has indeed been repositioned on the well after insertion of this sample.

an internal barcode reader 14 making it possible to identify the well plate 4 when a barcode is attached to it.

The drawer 2 can move only along the Y axis (front/rear) 15, which makes it possible to center the assembly 3 under the harpoon 12 to remove and reposition, specifically, a cap 6 from a column of the cap adapter 5.

To place an assembly 3 in this drawer 2, the removable element 8 is removed and this assembly 3 is inserted, then the removable element 8 is replaced. Once said removable element 8 has been closed, only the caps 6 and the adapter 5 remain visible. The plate is held in position by the removable element 8 of the drawer 2.

When a PCR plate is used instead of a deep-well plate, adding a riser keeps the PCR plate in contact just below the cap adapter.

The harpoon 12 can move on an X axis (left/right) 16 and a Z axis (up/down) 17 and can only remove or replace one cap 6 at a time. The movement of the harpoon 12 along the X axis makes it possible to specifically remove a cap 6 from a row of the adapter 5. It moves in a known manner on the axes 16 and 17 arranged above the drawer 2, in the sampling housing 1.

The drawer 2 of the sampling housing 1 is in the closed (internal) position when the harpoon 12 is to remove a cap 6 from the cap adapter. To this end, the movements of the harpoon along the X axis and of the drawer along the Y axis make it possible to remove a specific cap. The action of the harpoon along the Z axis makes it possible to remove the cap 6 from the cap adapter 5. The drawer 2 then opens to reveal only one open location, all the other wells being closed, thus avoiding any inter-well contamination. After inserting the sample into the well in question, the drawer is closed and the cap is replaced.

Thus, the drawer 2 is movable between a first position in which it is retracted into the sampling housing 1, placed under the harpoon 12 and out of reach of the operator, and a second position in which it has slid toward the operator so that the latter can introduce a sample into the only open well of the well plate 4, without risk of contamination and without risk of positioning error.

The sampling housing 1 is small in size to contain only a minimum of mechanical elements related to the movements of the drawer and the harpoon. The sampling housing can thus be connected to a control station composed of a computer part and an electronic part: the computer part makes it possible to control the actions of the sampling housing 1 according to a defined processing sequence. It is connected to a barcode scanner system in order to ensure the traceability of the samples and to reference the position of each sample in each of the 96 locations of the well plate 4 (creation of the plate plan). A photographic bench can also be connected to it in order to take a photo of the samples before sampling. By way of example, said computer part can be configured so that when a sample from a crime scene, for example, or a sample taken from a patient as part of COVID-19 screening, must be sampled, the operator scans the identifier of said sample, which will initiate a processing sequence (predefined by the operator) and the movement of the harpoon will open a well where the sample can be deposited by the operator after the device supporting the assembly is moved in front of the operator. It is also possible in the computer part to define the order (by columns, by rows, etc.) in which the wells will be opened one by one, and to define “prohibited” wells where no sample will be present, and whose caps will therefore not be opened by the sampling housing according to the invention;

the electronic part makes it possible to integrate the electronic cards and components necessary for the operation of the sampling housing 1. For example, a pedal can be connected to it that will actuate the harpoon 12 for removing and replacing the caps 6 in order to work in “hands-free” mode. The operator can thus reserve the use of both of his hands to handle the samples without having to touch the controller, thus avoiding any risk of contamination of the device during its use.

The sampling housing therefore forms a modular assembly with the control station.

The sampling housing (1) can be integrated into the control station.

The modular controller can then comprise a control station that incorporates a sampling housing (1).

In an alternative version, the modular controller can be formed by the sampling housing (1), which is connected to the control station, thus forming two separate assemblies. In this version, said sampling housing and the control station are connected and form the modular controller, but said sampling housing and said control station are separate from each other, said control station not integrating said sampling housing.

As will be understood by those skilled in the art, the sampling housing is as compact as possible to be positioned in a type 2 biosafety cabinet (BSC) or any other type of safety cabinet making it possible to protect the operator in relation to the nature of the sample. This compact aspect of the sampling housing according to the present invention also makes it possible to position it in a mobile laboratory or on any laboratory bench.

Thus, preferably, each side of said sampling housing has a dimension of less than 40 cm.

1. Sampling housing for biological samples, solid or liquid, comprising a device supporting an assembly consisting of a well plate, a cap adapter comprising as many orifices as there are wells and arranged on said well plate, and caps, each cap cooperating with a single well of the plate, characterized in that the device supporting the assembly is a drawer.

2. Sampling housing according to claim 1, characterized in that it comprises means for sliding the drawer in and out of said sampling housing that are coordinated with means for removing and replacing one cap of a well at a time.

3. Sampling housing according to claim 2, characterized in that the drawer is movable and is configured to slide between a position internal to said sampling housing in which a cap is removed or replaced, and a second position external to said sampling housing in which a sample to be analyzed is introduced into the well whose cap has been removed.

4. Sampling housing according to claim 2, characterized in that said means for removing and replacing a cap from one well at a time comprise a harpoon.

5. Sampling housing according to one of the preceding claims, characterized in that the drawer is composed of two parts: a base making it possible to receive the assembly and a removable element making it possible to hold said plate on said base.

6. Sampling housing according to claim 1, characterized in that it has a small footprint and is configured to be installed in a biosafety cabinet or a mobile laboratory.

7. Sampling housing according to claim 6, characterized in that each side of said sampling housing has a dimension of less than 40 cm.

8. Sampling housing according to claim 1, characterized in that it is configured to be connected to a control station.

9. Control station configured to be connected to a sampling housing according to claim 1, characterized in that it is composed of a computer part and an electronic part necessary for the operation of said sampling housing.

10. Modular controller, characterized in that it comprises a control station according to claim 9 that incorporates a sampling housing according to claim 1.

11. Modular controller, characterized in that it comprises a sampling housing according to claim 1 which form two distinct assemblies in said modular controller.

12. Method for sampling biological samples, solid or liquid, using a modular controller according to claim 10, comprising the steps of:
a. Scanning a solid or liquid biological sample;
b. Moving means for removing and replacing a cap of a well above a well of the plate;
c. Removing the cap from said well of step b. via said means for removing and replacing a cap of a well;
d. Moving, via the sampling housing, the device supporting the assembly in front of an operator;
e. Depositing, by an operator, of a biological sample in said open well;
f. Moving, via said sampling housing, the device supporting the assembly toward the inside of said sampling housing;
g. Closing, via said means for removing and replacing a cap, the open well comprising a sample by means of its cap.

13. Method for sampling biological samples, solid or liquid, according to claim 12, characterized in that steps a. to g. are repeated in order to successively sample biological samples in different wells, step c. removing a different cap each time.
